(19) United States
(12) Patent Application Publication
(10) Pub. No.: US 2011/0160539 A1
(43) Pub. Date: Jun. 30, 2011

(54) EXPANDABLE MEMBER DISSECTION PORT

(75) Inventor: David W. Robertson, Framingham, MA (US)

(73) Assignee: Boston Scientific Scimed, Inc.

(21) Appl. No.: 12/979,628

(22) Filed: Dec. 28, 2010

Related U.S. Application Data
(60) Provisional application No. 61/290,809, filed on Dec. 29, 2009.

(30) Foreign Application Priority Data
Dec. 23, 2010 (US) PCT/US10/62025

(51) Int. Cl. A61B 1/32

(52) U.S. Cl. 600/204

(57) ABSTRACT

Embodiments of the invention include a device to provide working access to a surgical site in a patient. The device may include a port component including an elongate member configured to pass through an opening in the patient. The elongate member may include a bore extending between a distal end and a proximal end of the elongate member. The port component may also include an expandable member disposed on the elongate member that may perform dissection and may form a seal to maintain insufflation pressure in a working space. The device may also include an insert component configured to be removably received inside the bore in the elongate member of the port component. The insert component may include at least one lumen that may removably receive at least one working instrument.

This application claims the benefit of priority from U.S. Provisional Application No. 61/290,809, filed Dec. 29, 2009, and PCT International Patent Application No. PCT/US10/62025 filed Dec. 23, 2010, which are herein incorporated by reference in their entirety.

### DESCRIPTION OF THE INVENTION

1. Field of the Invention

Embodiments of the present invention generally relate to surgical tools. Specifically, the present invention relates to a device for use in medical applications, such as for introducing a working port with a dissection balloon or other expandable member into a patient's body. Embodiments of the present invention also cover methods of using such devices. Those skilled in the art will recognize the benefits of applying the present invention to similar fields not discussed herein.

2. Background of the Invention

In general, it is desirable to minimize the invasiveness of medical procedures. These medical procedures may include therapeutic or diagnostic medical procedures. Invasive medical procedures are generally more expensive, and there is generally a greater risk of complication and discomfort for the patient. For example, open surgery, for a therapeutic or diagnostic purpose, is an invasive medical procedure with significant attendant risks. Since the performance of open surgery typically requires relatively large incisions, relatively large amounts of blood may be lost, the risk of infection may increase, and the potential for post-operative hernias may be higher. Furthermore, relatively large incisions require extended recovery times to allow the incisions to heal.

Laparoscopic procedures are generally less invasive than open surgery. Laparoscopic cholesystectomy (lap choly) is a laparoscopic procedure that involves incisions through the skin to access various body organs. For example, lap choly may involve access through a small incision in the skin and placement of a port into the peritoneal cavity to allow removal of an inflamed gall bladder. A working instrument may be introduced into the body through the port. The working instrument may be a flexible instrument, such as an endoscope, introduced into the body to further access the inside of the body. A surgeon may use ports and working instruments to perform any desired therapeutic or diagnostic procedure at a work site inside the body.

Although growing capabilities of devices such as endoscopes allow physicians to perform an increasing variety of surgeries through minimally invasive routes, further refinements may allow even less traumatic surgical access and/or performance of traditional open surgical or laparoscopic procedures. Accordingly, methods and devices that enhance access would be beneficial.

It is to be understood that both the foregoing general description and the following detailed description are exemplary and explanatory only and are not restrictive of the invention.

### SUMMARY OF THE INVENTION

In one aspect, a device to provide working access to a surgical site in a patient may include a port component including an elongate member configured to pass through an opening in the patient. The elongate member may include a bore extending between a distal end and a proximal end of the elongate member. The port component may also include an expandable member disposed on the elongate member. The device may also include an insert component configured to be removably received inside the bore in the elongate member of the port component. The insert component may include at least one lumen.

In another aspect, a method to provide working port access to a surgical site in a patient may include advancing an expandable member in a collapsed configuration and an elongate member through an opening in the patient. The expandable member may be disposed on the elongate member. The method may also include expanding the expandable member to an expanded configuration to separate tissue layers in the patient and inserting a removable insert component including at least one lumen into a bore in the elongate member.

In a further aspect, a method to provide working port access to a surgical site in a patient may include advancing an expandable member in a collapsed configuration through an opening in the patient. The expandable member may be disposed on an elongate member. The method may also include expanding the expandable member to an expanded configuration, positioning the expandable member in the expanded configuration to abut tissue of the patient to form a seal between the expandable member and the tissue of the patient, and directing insufflation fluid through at least one of the elongate member and an insert component that is removably received in the elongate member so that the insufflation fluid forms a working space in the patient.

In yet another aspect, a method to provide working port access to a surgical site in a patient may include advancing an expandable member in a collapsed configuration and an elongate member through an opening in the patient. The expandable member may be disposed on the elongate member. The method may also include expanding the expandable member to an expanded configuration to separate tissue layers in the patient, removing a first insert component from inside of the elongate member without removing the expandable member from inside the patient, and inserting a second insert component into the elongate member when the expandable member is inside the patient. The first insert component may have a different structure than the second insert component.

Additional objects and advantages of the invention will be set forth in part in the description which follows, and in part will be obvious from the description, or may be learned by practice of the invention. The objects and advantages of the invention will be realized and attained by means of the elements and combinations particularly pointed out below.

It is to be understood that both the foregoing general description and the following detailed description are exemplary and explanatory only and are not restrictive of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a side view of a port component and an insert component of a dissection port, according to an exemplary embodiment of the invention;

FIG. 2 is a perspective view of a port component of a dissection port, according to an exemplary embodiment of the invention;

FIG. 3 is a cross-sectional view of the port component of FIG. 2;

FIGS. 4 and 5 are perspective views of insert components of a dissection port, according to exemplary embodiments of the invention;

FIGS. 6a, 6b, 7, and 8 are cross-sectional views of insert components of a dissection port, according to exemplary embodiments of the invention;

FIGS. 9-13 are perspective views of a port component, an insert component, and various working instruments of a dissection port, according to exemplary embodiments of the invention;

FIG. 14 is a cross-sectional view of a dissection port and an opening in an abdominal wall of a patient, according to an exemplary embodiment of the invention;

FIG. 15 is a cross-sectional view of the distal end of the dissection port of FIG. 14 inserted into the opening in the patient;

FIG. 16 is a cross-sectional view of an expandable member of the dissection port of FIG. 14 expanded to separate part of the peritoneum from the overlying layer in the patient;

FIG. 17 is a cross-sectional view of the expandable member of the dissection port of FIG. 14 expanded to create a working space in the patient;

FIG. 18 is a cross-sectional view of the dissection port of FIG. 14 with an interchangeable insert component; and

FIGS. 19-22 are cross-sectional views showing the movement of an elongate member with an expandable member of a dissection port, according to an exemplary embodiment of the invention.

### DESCRIPTION OF THE EMBODIMENTS

Reference will now be made in detail to exemplary embodiments of the invention, examples of which are illustrated in the accompanying drawings. Wherever possible, the same reference numbers will be used throughout the drawings to refer to the same or like parts.

The terms “proximal” and “distal” are used herein to refer to the relative positions of the components of the exemplary dissection port 10. When used herein, “proximal” refers to a position relatively closer to the exterior of the body or closer to the surgeon using the dissection port 10. In contrast, “distal” refers to a position relatively further away from the surgeon using the dissection port 10 or closer to the interior of the body.

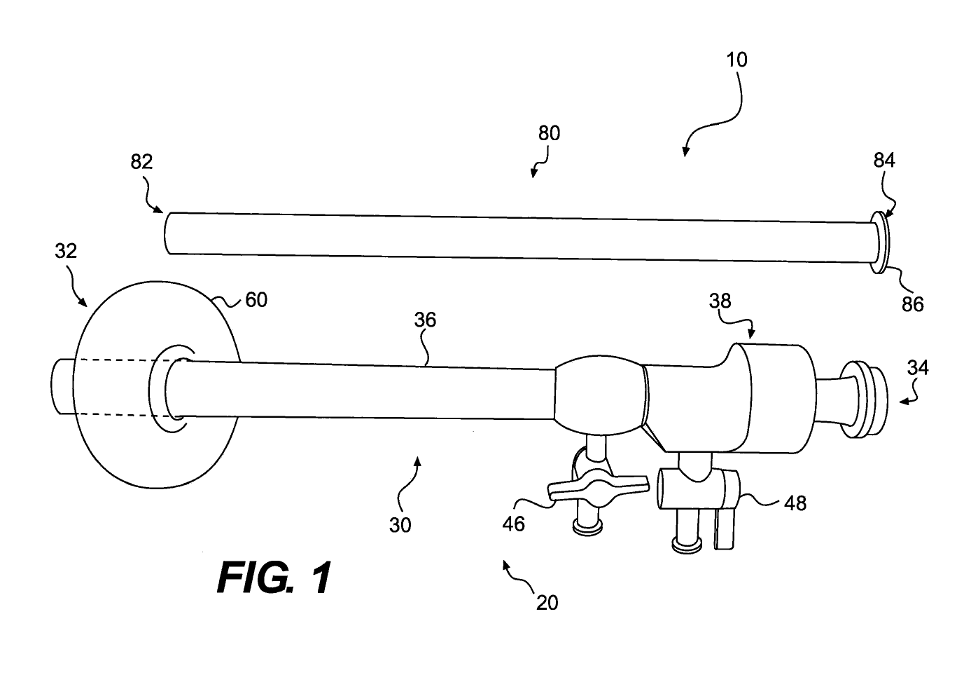

FIG. 1 depicts an exemplary dissection port 10 that may be used for any therapeutic or diagnostic endoscopic procedure. The phrase “endoscopic procedure” is broadly used to indicate any medical procedure that may be performed by inserting an endoscope, guide tube, catheter, or any other medical device into the body through any anatomic opening. Although in the description that follows, the dissection port 10 is described and shown as being inserted into the body through the abdominal wall, it should be emphasized that this description is exemplary only. In general, embodiments of the current disclosure may be applicable to any application where a medical device is inserted into the body through an anatomic opening (e.g., an incision or a natural orifice). For example, embodiments of the current disclosure may be used in natural orifice transluminal endoscopic surgical (NOTES) procedures or single incision laparoscopic surgical (SILS) procedures.

In a NOTES procedure, a working instrument may be introduced into the body through a body orifice (e.g., mouth, anus, nose, urethra, vagina, etc.). Therefore, a NOTES procedure may allow access to various body organs through an incision in a luminal wall without having to puncture the skin. After one or more working instruments pass through the natural orifice and are positioned at a desired work site within the body, the surgeon may perform any desired therapeutic or diagnostic procedure at the work site.

In a SILS procedure, the surgeon may create a single incision through the skin of the patient to access the desired work site. The incision acts as a single entry point. After one or more working instruments pass through the single entry point and are positioned at the desired work site in the body, the surgeon may perform any desired therapeutic or diagnostic procedure at the work site.

The dissection port 10 may be used for other procedures, such as, but are not limited to, procedures for single access site (SAS) laparoscopic surgery, single port access (SPA) surgery, single port laparoscopy (SPL), single site access (SSA) surgery, one-port umbilical surgery (OPUS), visibly scarless urologic surgery (VSUS), single laparoscopic port procedure (SLiPP), natural orifice trans umbilical surgery (NOTUS), trans umbilical endoscopic surgery (TUES), trans umbilical laparoscopic assisted (TULA) surgery, embryonic natural orifice transluminal endoscopic surgery (E-NOTES), single-incision multiport laparoendoscopic surgery (SIMPLE), laparo-endoscopic single site surgery (LESS), and single port incisionless conventional equipment-utilizing surgery (SPICES).

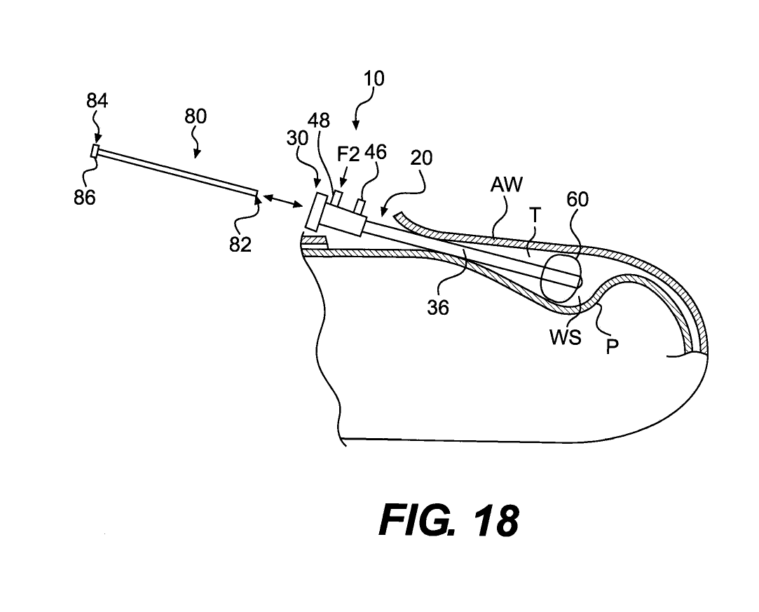

According to an embodiment, the dissection port 10 may include a port component 20 and an insert component 80 that may be inserted into the port component 20. As described below, the dissection port 10 may be inserted into a patient to separate or dissect tissue layers in the patient. By separating the tissue layers, the dissection port 10 may create a working space WS (FIGS. 17 and 18) between the tissue layers, and the dissection port 10 may direct insufflation fluid into the working space WS to maintain the separation of the tissue layers. The dissection port 10 may be made of any suitable material capable of being inserted into the body, e.g., a suitable biocompatible material.

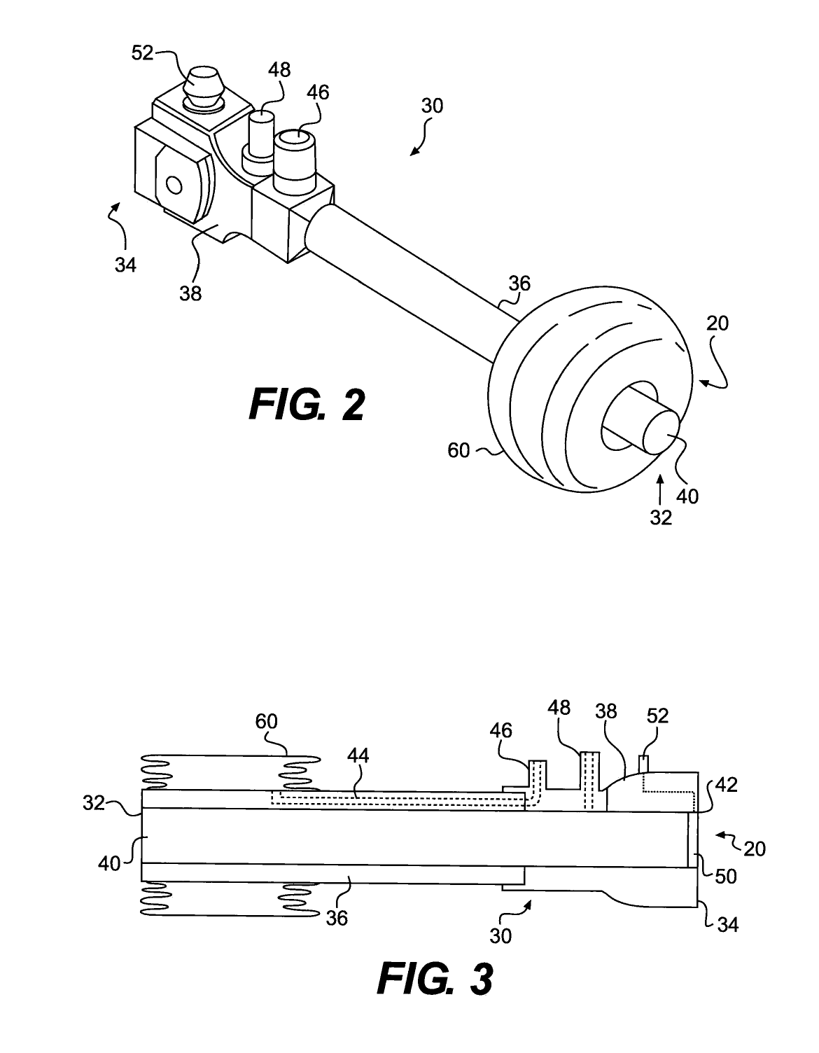

FIGS. 2 and 3 depict the port component 20, according to an exemplary embodiment. The port component 20 may include an elongate member 30 and an expandable member 60.

The elongate member 30 may include a distal end 32 and a proximal end 34, and may be rigid, malleable, or flexible. The elongate member 30 may also include a cannula 36, a housing 38, and a hollow cavity or bore 40 extending through the cannula 36 and the housing 38. The housing 38 may be disposed proximal to the cannula 36 and may include one or more valves or other devices that may be controlled by the surgeon or other user.

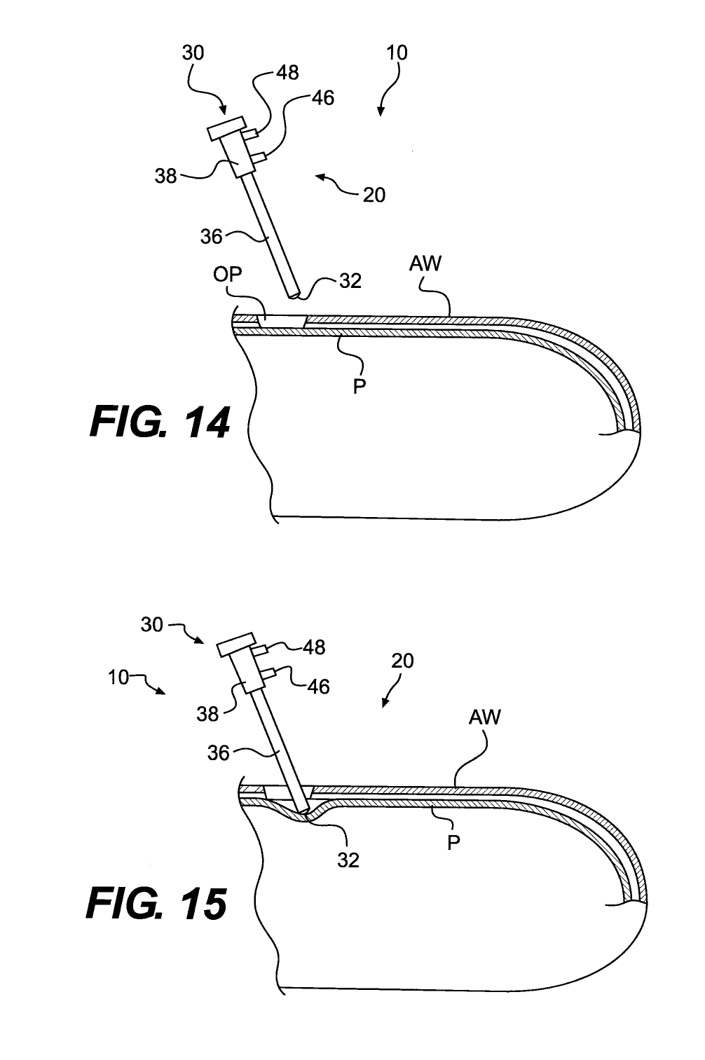

The cannula 36 may be a tubular member configured to be at least partially inserted into an opening OP (FIG. 14) in the patient, such as a natural orifice in the body (e.g., mouth, rectum, anus, nose, urethra, umbilicus, vagina, etc.) or an incision created by the surgeon. The elongate member 30 may be advanced through the opening in the patient so that the distal end 32 may be positioned at or near the working space WS.

The bore 40 may extend longitudinally (axially) between the distal end 32 and the proximal end 34 of the elongate member 30. As shown in the embodiment of FIG. 3, the bore 40 may extend from an opening at a distal end of the cannula 36 to a port opening 42 in the housing 38 at the proximal end 34 of the elongate member 30. The insert component 80 may be inserted into the bore 40.

Although the elongate member 30 shown in FIGS. 2 and 3 includes a single bore 40 extending through the distal end 32 and proximal end 34 of the elongate member 30, one or more additional bores or lumens (not shown) extending through the distal end 32 and/or the proximal end 34 of the elongate member 30 may be included. For example, one or more of an aspiration lumen, an irrigation lumen, an illumination lumen, a viewing lumen, and a working lumen, may run longitudinally through the elongate member 30.

The elongate member 30 may include one or more lumens extending longitudinally through the elongate member 30, and such lumen(s) may or may not extend through the distal end 32 and/or the proximal end 34 of the elongate member 30. For example, in the embodiment shown in FIG. 3, the elongate member 30 may include an inflation lumen 44 extending longitudinally through the elongate member 30, but not extending through the distal end 32 or the proximal end 34 of the elongate member 30.

The inflation lumen 44 is a cavity in the elongate member 30 through which a fluid, such as a liquid or gas, may pass to expand (inflate) and contract or collapse (deflate) the expandable member 60. For example, the inflation fluid may be air, water, carbon dioxide, or saline solution. As shown in FIG. 3, the inflation lumen 44 may be fluidly connected at one end to a first valve 46 disposed on the housing 38 and at an opposite end to the expandable member 60. The inflation lumen 44 may extend through the housing 38 and the cannula 36 of the elongate member 30. Alternatively, the inflation lumen 44 may extend through the cannula 36 only, and the first valve 46 may be disposed on the cannula 36. The first valve 46 may permit fluid to enter the inflation lumen 44 and may prevent fluid from exiting from inside the inflation lumen 44. The first valve 46 may also allow the surgeon to vent fluid from inside the inflation lumen 44 and the expandable member 60.

For example, a source of the inflation fluid, such as a pump or syringe, may be connected to the first valve 46 to direct inflation fluid into the inflation lumen 44 and the expandable member 60. After the expandable member 60 is expanded to a desired expanded configuration, the source of the inflation fluid may be disconnected from the first valve 46, and then the first valve 46 may prevent the inflation fluid from exiting the inflation lumen 44 and the expandable member 60. After removing the source of the inflation fluid, the surgeon may also control the first valve 46 to release or vent inflation fluid from the inflation lumen 44 and the expandable member 60.

The phrase “expandable member” is used in a broad sense to denote any expandable structure, such as a balloon or other inflatable structure, regardless of the elasticity of the material comprising the structure. For example, the phrase “expandable member” may denote a thin-walled structure made of material of low elasticity (which does not stretch significantly during inflation) or highly elastic material (which does stretch significantly during inflation). For example, the expandable member 60 may be made from polyethylene terephthalate (PET), polyurethanes, polyethylenes and ionomers, copolyesters, rubbers, polyamides, silicone, latex, or any other suitable materials known in the art. The expandable member 60 may be mechanically, electrically, pneumatically or hydraulically expanded and collapsed without departing from the scope of the invention.

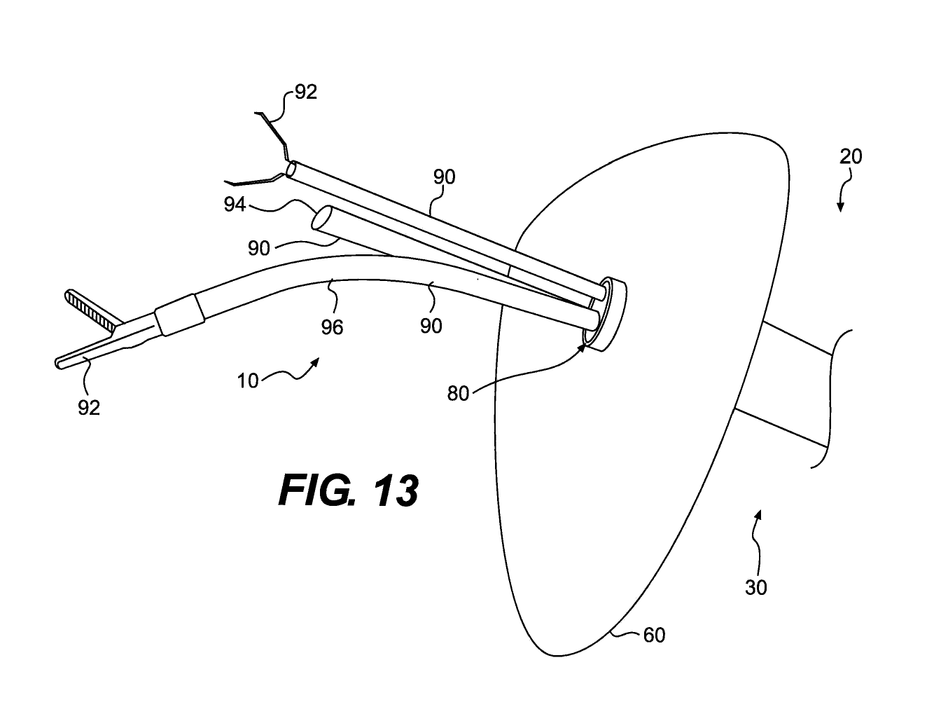

FIG. 2 shows an exemplary embodiment of the expandable member 60 in an expanded configuration, and FIG. 3 shows the expandable member 60 in a collapsed configuration. The particular expanded exterior configuration of the expandable member 60, such as the volume, width, depth, radius, length, or other dimension, may be selected depending on the particular circumstances of use. For example, in the embodiment shown in FIGS. 1-3 and 9-12, the expandable member 60 in its expanded configuration may be toroidal or doughnut-shaped. Alternatively, the expandable member 60 may be spherical or cylindrical (e.g., extending farther along the length of the cannula 36). As shown in FIGS. 1-3 and 9-13, the expandable member 60 may include a hole through the center, through which the cannula 36 passes through and is attached, and the outer profile of the expandable member 60 may be circular (FIGS. 1-3 and 9-12), oval, elliptical, teardrop-shaped, triangular (FIG. 13), square, etc.

The housing 38 may include a second valve 48 fluidly connected to the bore 40 extending through the housing 38 of the elongate member 30, as shown in FIGS. 2 and 3. Alternatively, when the insert component 80 is inserted into the bore 40, the second valve 48 may be fluidly connected to one or more lumens 88 in the insert component 80 instead of, or in addition to, being fluidly connected to the bore 40. Alternatively, the second valve 48 may be disposed on the cannula 36. The second valve 48 may permit fluid to enter the bore 40 or the lumen(s) 88 in the insert component 80, and may prevent fluid from exiting the bore 40 or the lumen(s) 88. The second valve 48 may also allow the surgeon to vent fluid from inside the bore 40 or the lumen(s) 88.

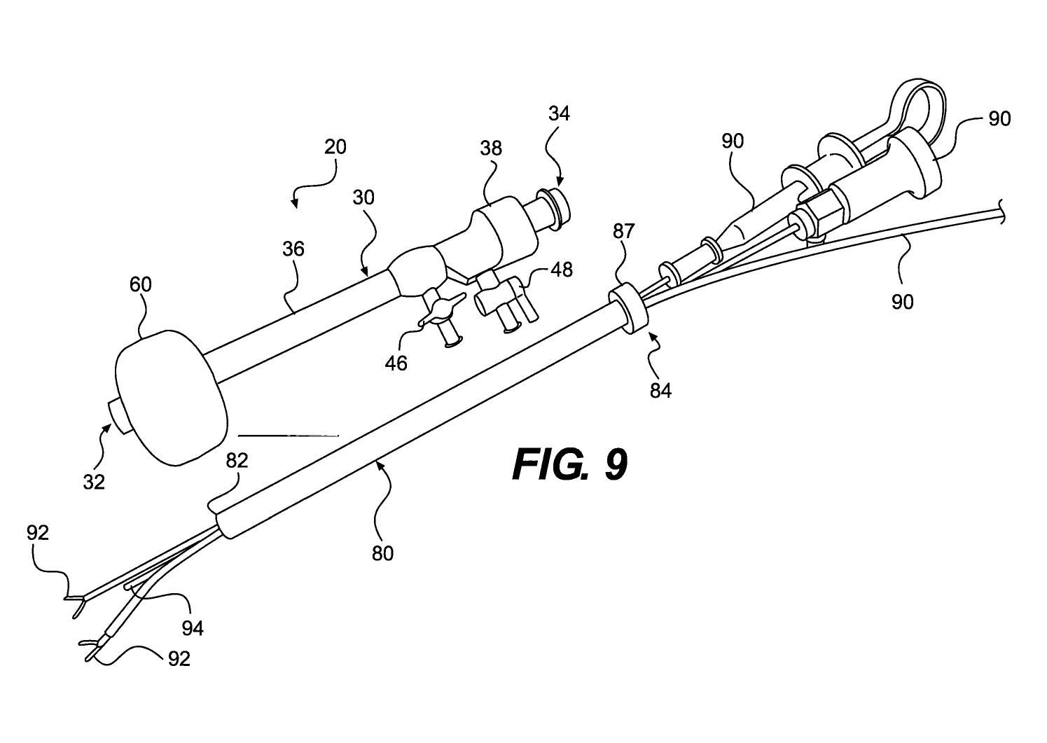

For example, insufflation fluid may be directed through the second valve 48, and through the bore 40 or the lumen(s) 88 in the insert component 80 placed in the bore 40. The insufflation fluid may be supplied to the working space WS (FIGS. 17 and 18), for example, to maintain or further extend the separation between tissue layers after dissecting the tissue layers. The insufflation fluid may be a liquid or gas, such as air, water, carbon dioxide, or saline solution. A source of the insufflation fluid, such as a pump or syringe, may be connected to the second valve 48 to direct the insufflation fluid into the bore 40 or the lumen(s) 88 in the insert component 80, which directs the insufflation fluid through a respective opening of the bore 40 or the lumen(s) 88 at the distal end 32 of the elongate member 30. After providing a desired amount of insufflation fluid (for example, based on observing the working space WS using an optical device 94 (FIGS. 9-13)), the source of the insufflation fluid may be disconnected from the second valve 48. After removing the source of the insufflation fluid, the second valve 48 may prevent the insufflation fluid from exiting the bore 40, the lumen(s) 88 in the insert component 80, and/or the working space WS. Also, after removing the source of the insufflation fluid, the surgeon may control the second valve 48 to release or vent insufflation fluid from the bore 40, the lumen(s) 88 in the insert component 80, and/or the working space WS.

As shown in FIG. 3, the housing 38 may also include a third valve or seal 50 located at or near the port opening 42, and fluidly connected to the bore 40. Alternatively, the third valve 50 may be disposed in the cannula 36. The third valve 50 may permit fluid to enter the bore 40 and may prevent fluid from exiting the bore 40.

The housing 38 may also include a button 52 or other user-controlled device connected to the third valve 50. According to an embodiment, the surgeon may push the button 52 to open the third valve 50 so that the insert component 80 may be inserted into the bore 40. After releasing the button 52, the third valve 50 may close, thereby preventing fluids or other substances from exiting the bore 40.

Each of the first valve 46, the second valve 48, and the third valve 50 may be any type of suitable valve known to those skilled in the art for controlling the flow of fluid therethrough, such as a flexible diaphragm, membrane, septum, elastomeric seal, or other flow control device.

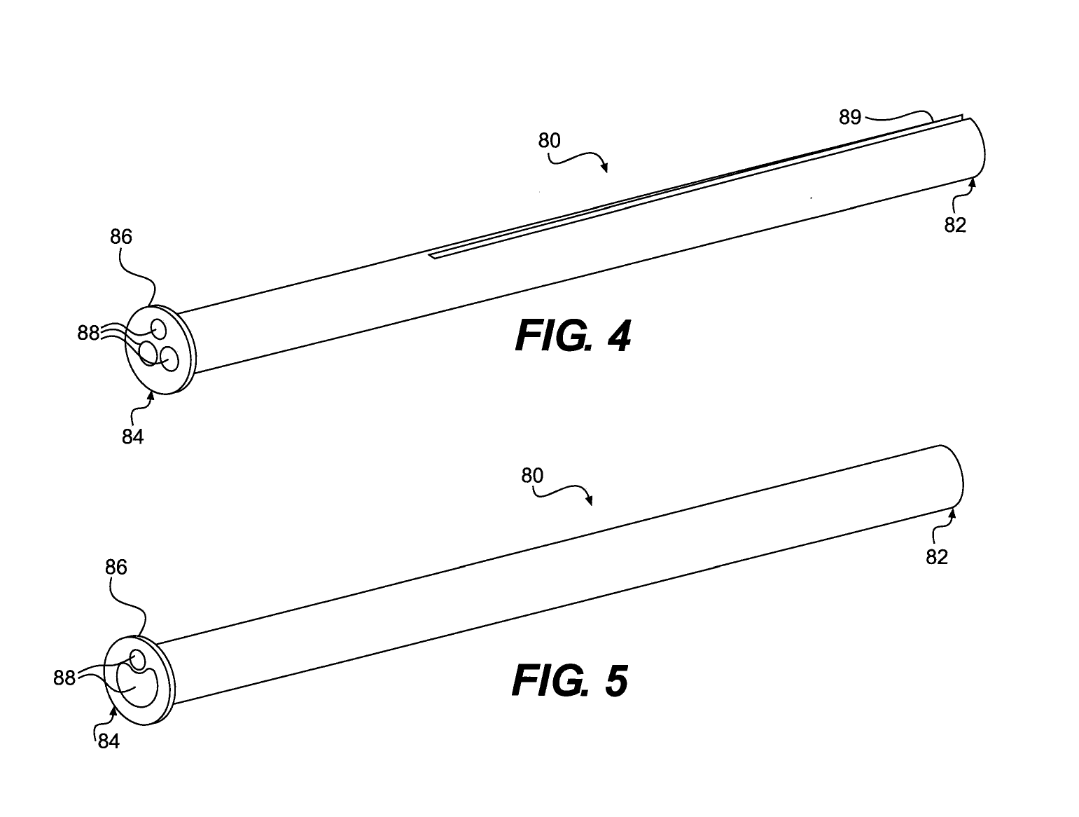

FIGS. 2 and 3 show that the housing 38 is provided proximally to the cannula 36 so that respective bores extending through the cannula 36 and the housing 38 align to form the bore 40 extending through the elongate member 30. Alternatively, the cannula 36 may extend longitudinally between the distal end 32 and the proximal end 34 of the elongate member 30, and the housing 38 may surround a portion of the cannula 36 near a proximal end of the cannula 36. In such an embodiment, the second valve 48 may extend through the cannula 36 to allow fluid to enter the bore 40 or the lumen(s) 88 in the insert component 80, e.g., through a hole in a wall of the insert component 80. Additional space for fluid flow between the bore 40 and the insert component 80 may be provided, e.g., by at least one longitudinal or spiral groove 89 on an outer surface of the insert component 80 or using one or more spacers on an inner surface of the bore 40. FIG. 4 shows an exemplary embodiment of the insert component 80 that includes the groove 89 that is capable of receiving fluid from the second valve 48 when the insert component 80 is placed inside the bore 40 in the elongate member 30 and that extends to the distal end 82 of the insert component 80. Alternatively, the groove 89 may be omitted from the insert component 80 shown in FIG. 4. Also, the port opening 42 may be provided at the proximal end of the cannula 36 (instead of the housing 38), and the third valve 50 may be provided at the port opening 42 in the cannula 36.

FIGS. 4, 5, 6a, 6b, 7, and 8 depict the insert component 80, according to various exemplary embodiments. The insert component 80 is configured to be slidably inserted into the bore 40 in the elongate member 30, and may be rigid, malleable, or flexible. For example, both the elongate member 30 and the insert component 80 may be malleable and/or flexible so that when the insert component 80 is inserted into the elongate member 30, the elongate member 30 and the insert component 80 may be bent or shaped, e.g., to follow a curving path in the patient's body, to direct or steer the distal ends 32, 82 of the elongate member 30 and the insert component 80, etc. The insert component 80 may include a distal end 82 and a proximal end 84. The insert component 80 may also include a flange 86 at the proximal end 84 of the insert component 80. When the insert component 80 is disposed inside the bore 40 in the elongate member 30, the flange 86 may abut the proximal end 34 of the elongate member 30 to hold the insert component 80 in place longitudinally.

One or more lumens 88 may extend longitudinally through the insert component 80. The lumens 88 may extend through the distal end 82 and the proximal end 84 of the insert component 80. The lumens 88 may include one or more of an aspiration lumen, an irrigation lumen, an illumination lumen, a viewing lumen, a working lumen, etc. The structure or configuration of the lumens 88, e.g., the number, location, size, and shape of the lumens 88 and a shape of the distal end 82 of the insert component 80, may depend on the particular circumstances of use for the dissection port 10 when the dissection port 10 includes the particular insert component 80. For example, in the embodiment shown in FIGS. 4, 6b, and 7 the insert component 80 includes three lumens, and in the embodiments shown in FIGS. 5, 6a, and 8, the insert component 80 includes two lumens. Alternatively, the insert component 80 may include zero lumens, one lumen, or more than three lumens.

According to an embodiment, the insert component 80 may include zero lumens. For example, the insert component 80 may include a blunt and/or rounded tip at the distal end 82 of the insert component 80 (e.g., an obturator). In such an embodiment, the rounded tip may be used to separate tissue layers when the distal end 32 of the elongated member 30 is inserted into the patient, as described below and shown in FIG. 15. Alternatively, the insert component 80 may include at least one partial lumen, such as a lumen that does not extend through the distal face of the insert component 80. For example, the partial lumen may extend towards a window or other opening near the distal end, e.g., on a circumferential surface or other outer surface of the insert component 80, and an optical device, e.g., a rigid optical device as described below, may be inserted into the partial lumen and positioned near the window.

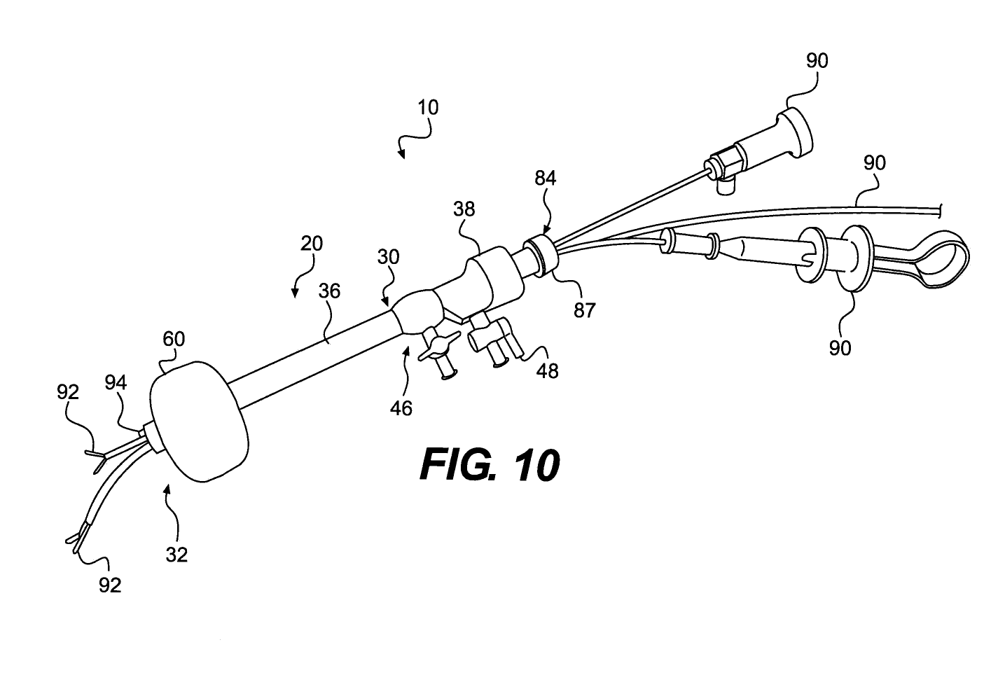

The insert component 80 may include an attached or removable seal 87 (FIGS. 9 and 10) disposed on the proximal end 84 of the insert component 80. Each lumen 88 may include a separate seal 87. The seal 87 may permit fluid to enter the lumen(s) 88 in the insert component 80 and may prevent fluid from exiting the lumen(s) 88 through the proximal end 84 of the insert component 80 with or without a suitable working instrument 90 (FIGS. 9-13) passed through the lumen 88. The seal 87 may be any type of suitable seal known to those skilled in the art for sealing fluid therethrough, such as a flexible diaphragm, membrane, septum, elastomeric seal, or other flow control device.

An optical device (such as the optical device 94 shown in FIGS. 9-13) or other sensor device may be embedded into the insert component 80 at or near the distal end 82 of the insert component 80, e.g., mounted on the distal face. The optical device may include an illumination component, and a camera, lens, digital imaging chip, or other image receiving device, which may transmit (e.g., wirelessly, or using wires or fiber optics embedded along the length of the insert component 80) an image or other signal to a signal processing device, a recorder, or a monitor or other display device viewable by the surgeon. Alternatively or in addition, the sensor device may monitor and transmit other characteristics, such as temperature, pressure, pH, etc.

The dissection port 10 may be provided with a plurality of interchangeable insert components 80 having different configurations of one or more lumens 88, or no lumens. The surgeon may replace the insert components 80 at any time when the dissection port 10 is outside the patient or when the port component 20 has been inserted into the patient, e.g., when the distal end 32 of the elongate member 30 is inserted into the patient or during the process of insertion, as described below. For example, during a surgical procedure and when the port component 20 is inserted into the surgeon, the surgeon may change insert components 80 based on which insert component 80 is better suited for an intended task. The insert component 80 may be changed (thereby changing the configuration of lumens 88 in the dissection port 10) without having to remove the port component 20 from the patient.

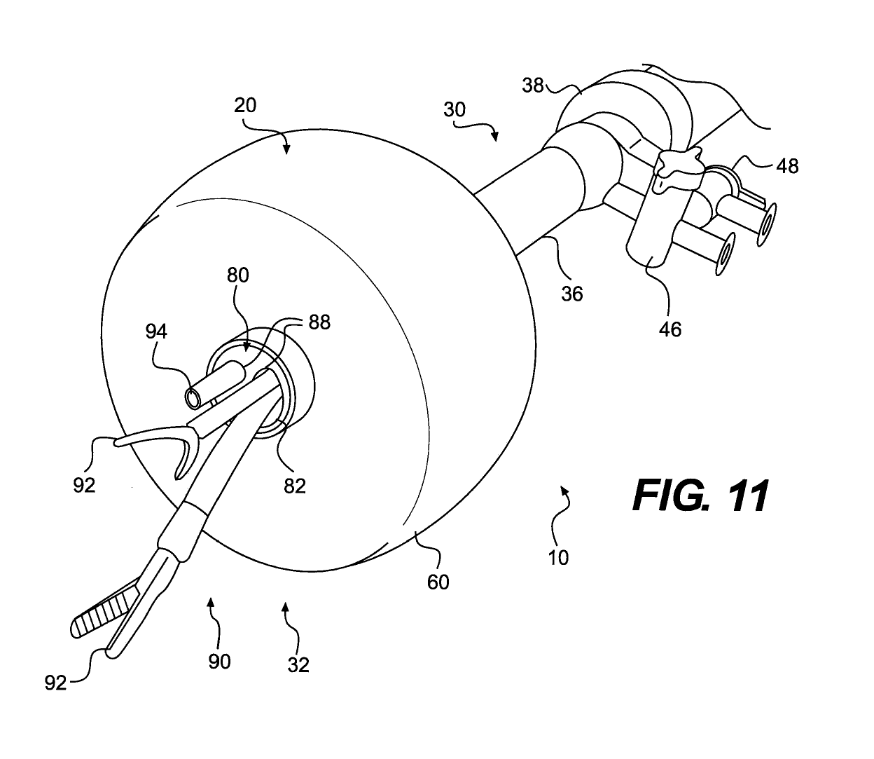

FIGS. 9-13 depict the dissection port 10 including a plurality of working instruments 90 inserted through the insert component 80. One or more working instruments 90 may be placed simultaneously into any lumen 88 of the insert component 80. Additionally, one or more additional properly dimensioned insert components 80 may be removeably placed in any lumen 88 of the insert component 80. FIG. 9 shows exemplary working instruments 90 inserted into the lumens 88 in the insert component 80, which is configured to be inserted into the port component 20. FIGS. 10-13 show exemplary working instruments 90 inserted into the lumens 88 in the insert component 80, which is inserted into the port component 20. In the exemplary embodiment shown in FIG. 11, the insert component 80 includes two lumens 88, and one working instrument 90 is inserted into one lumen 88 while two working instruments 90 are inserted into the other lumen 88. In the exemplary embodiment shown in FIGS. 9, 10, 12, and 13, the insert component 80 includes three lumens 88, and one working instrument 90 is inserted into each of the three lumens 88.

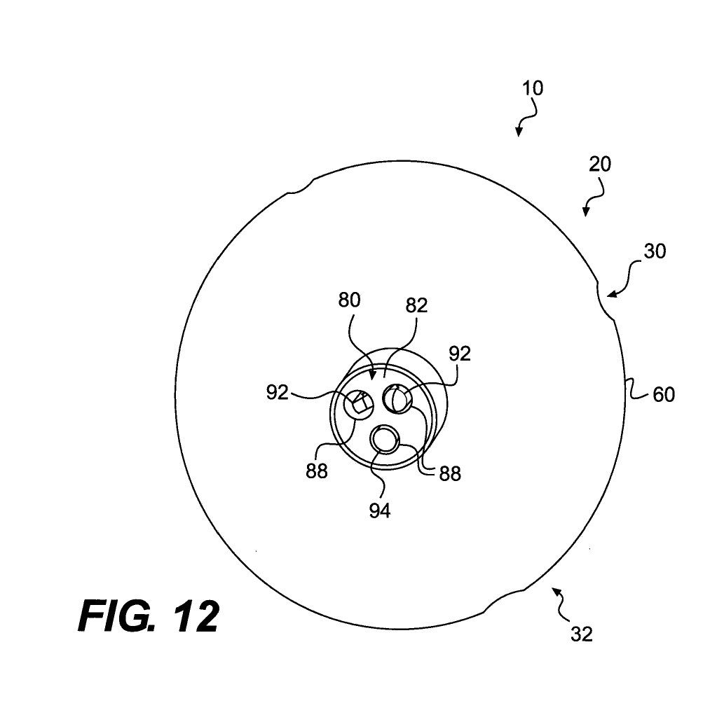

The surgeon may move each working instrument 90 longitudinally (e.g., in the distal and proximal directions, axially), laterally (e.g., side to side), and/or rotationally with respect to the dissection port 10. FIG. 12 shows the dissection port 10 with the working instruments 90 retracted longitudinally into the insert component 80. The working instruments 90 may be flexible, rigid, bent, straight, steerable, etc., and may include one or more lumens for additional working instruments, fluids, etc., to pass into the working space.

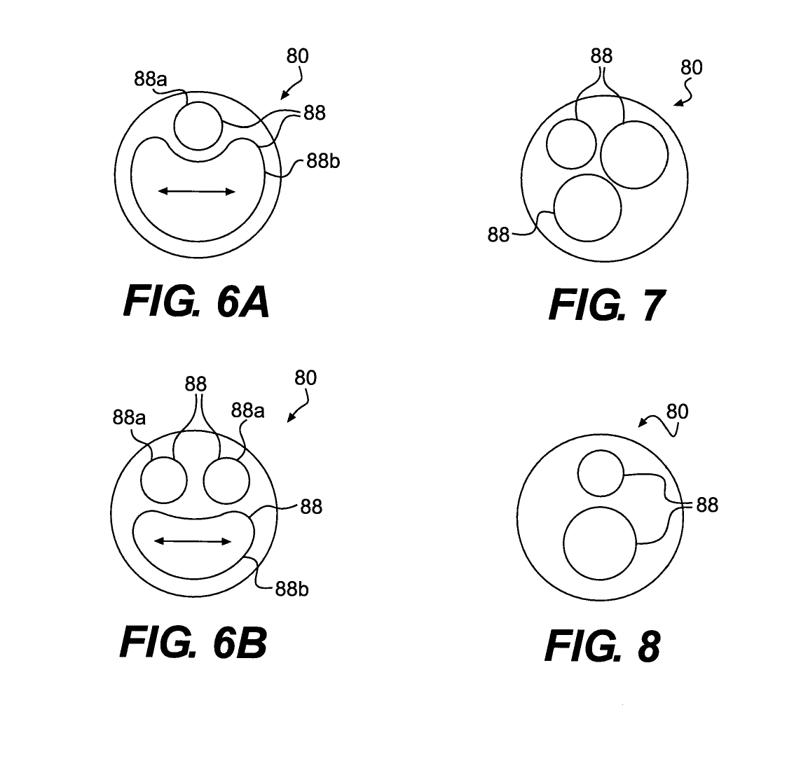

In exemplary embodiments, as shown in FIGS. 6a and 6b, at least one of the lumens 88a in the insert component 80 may be configured to hold at least one working instrument 90 relatively rigid in relation to the longitudinal axis of the port component 20 (e.g., limiting lateral and angular movement) while at least one other lumen 88b may be configured to allow lateral (e.g., in the direction shown by the arrows), angular, and/or rotational movement of at least one working instrument 90 inserted into the lumen 88b. The lumens 88a, 88b may include separate seals 87. The larger lumens 88b may be sized to allow for triangulation of the working instruments 90 inserted into the lumen 88b. For example, a first working instrument 90 inserted into the lumen 88b may hold tissue at one location while a second working instrument 90 inserted into the lumen 88b may retract tissue with respect to the tissue held by the first working instrument 90, while a third working instrument 90 may be used to cut tissue. As a result, the working instruments 90 inserted through the lumen 88b may approach the working space at different angles and may each move with respect to the other working instruments 90.

Each working instrument 90 may include an end effector 92 at a distal end of the working instrument 90. The end effector 92 may include, but is not limited to, a cutting device (e.g., scissors, forceps, tissue cutter, etc.), a fixation device, a manipulation device, a dissection device, a support device, a sealing device, a closure device (e.g., clips, staples, loops, ligator, suturing device, etc.), a retrieval device (e.g., snare, basket, loop, a fluid extraction device, etc.), a tissue exploration device (e.g., the optical device 94, etc.), a tissue sampling device, a delivery device, a device for aiding in the patency of a lumen or for dilating an opening (e.g., a balloon or other expandable member, patency brush, stent, fan retractor, wire structure, etc.), a grasping device, an active device such as a radiofrequency (RF) or ultrasonic cutter or sealer, an optical imaging device, etc. The working instrument 90 may include control wires or other devices connected to the end effector 92 to allow the surgeon to control the movement of the end effector 92. For example, as shown in FIG. 13, the end effector 92 may include an articulating portion 96, that provides a longitudinal, lateral, and/or rotational articulation, or manipulation of the end effector 92. For example, the end effector 92 may deliver a mesh layer or other substance to the working space, and control wires may be provided to control the movement, fixation, and release of the mesh layer or other substance into the patient using the articulation portion 96. Accordingly, the working instruments 90 and end effectors 92 may be any type of suitable working instruments and end effectors known to those skilled in the art.

Alternatively, the working instrument 90 may not include an end effector. For example, the working instrument 90 may include a blunt and/or rounded tip for exploration and/or for assisting another working instrument or end effector (e.g., an obturator). As another alternative, the working instrument 90 may include an open distal end for the delivery of a treatment fluid or solid and/or for collection of a bodily fluid or tissue sample.

As shown in FIGS. 9-13, the end effector 92 may include the optical device 94, such as the optical device described above that may be embedded or attached at the distal end 82 of the insert component 80. The optical device 94 may be embedded or attached to a distal end of the working instrument 90. The optical device 94 may be a working instrument 90, such as a rigid or flexible endoscope. The surgeon may move the working instrument 90 longitudinally, laterally, and/or rotationally with respect to the insert component 80 and the dissection port 10 to control the position of the optical device 94.

FIGS. 14-18 depict a method to provide surgical access to a site within a patient using the dissection port 10, according to an embodiment of the invention. For the purpose of illustration only, the method is described in the context of placing an access port through the abdominal wall by separating the tissue layers with balloon dissection to perform a surgical procedure inside the abdominal wall. Variations on the described embodiment (and in the apparatus employed to perform it) are useful for performing other medical procedures throughout the body.

As shown in FIG. 14, an incision, e.g., about 5-20 mm long, is made in the epithelium of an abdominal wall AW. Additional blunt or sharp dissection forms an opening OP. Alternatively, such as in a NOTES procedure, the opening OP is made after passing the dissection port 10 through a natural orifice, e.g., mouth, anus, nose, urethra, vagina, etc., and making an incision in a lumenial wall. Although not indicated in FIG. 14, the expandable member 60 is in the collapsed configuration.

As shown in FIG. 15, the distal end 32 of the elongated member 30 of the port component 20 is inserted into the opening OP to bring the distal end 32 of the elongated member 30 in the port component 20 into contact with the peritoneum P. The bore 40 in the elongated member 30 may be empty (without the insert component 80 inserted into the bore 40), or the insert component 80 may be placed in the bore 40. Although not indicated in FIG. 15, the expandable member 60 is in the collapsed configuration.

At any time during the steps shown in FIGS. 14-18, the insert component 80 with a blunt and/or rounded tip (an obturator) with or without optical imaging components may be inserted into the bore 40 in the elongated member 30, and the blunt and/or rounded tip of the insert component 80 may be brought into contact with the peritoneum P. The obturator may include zero lumens. Alternatively, the insert component 80 with one or more lumens 88 may be replaced and/or inserted into the bore 40 in the elongated member 30, and the working instrument 90 with a blunt and/or rounded tip (the obturator described above) may be inserted into one of the lumens 88 in the insert component 80. The blunt and/or rounded tip of the insert component 80 or the working instrument 90 may be brought into contact with the peritoneum P.

Additional pressure may be exerted on the proximal end 34 of the elongated member 30, the proximal end 84 of the insert component 80 with the blunt and/or rounded tip, and/or the proximal end of the working instrument 90 with the blunt and/or rounded tip, which presses against the peritoneum P, thereby detaching the part of the peritoneum in the immediate vicinity of the opening OP.

The obturator may also include the optical device 94 embedded into or on the distal end of the obturator. The obturator may also be removed and replaced by the insert component 80 with the optical device 94 at or near its distal end 82, or the working instrument 90 with the optical device 94 at or near its distal end. Alternatively, when the obturator is provided as a working instrument 90 in one of the lumens 88 in the insert component 80, another working instrument 90 with the optical device 94 may be provided in another lumen 88 in the insert component 80. As a result, the optical device 94 enables viewing of the space between the dissected tissue layers as the layers are separated.

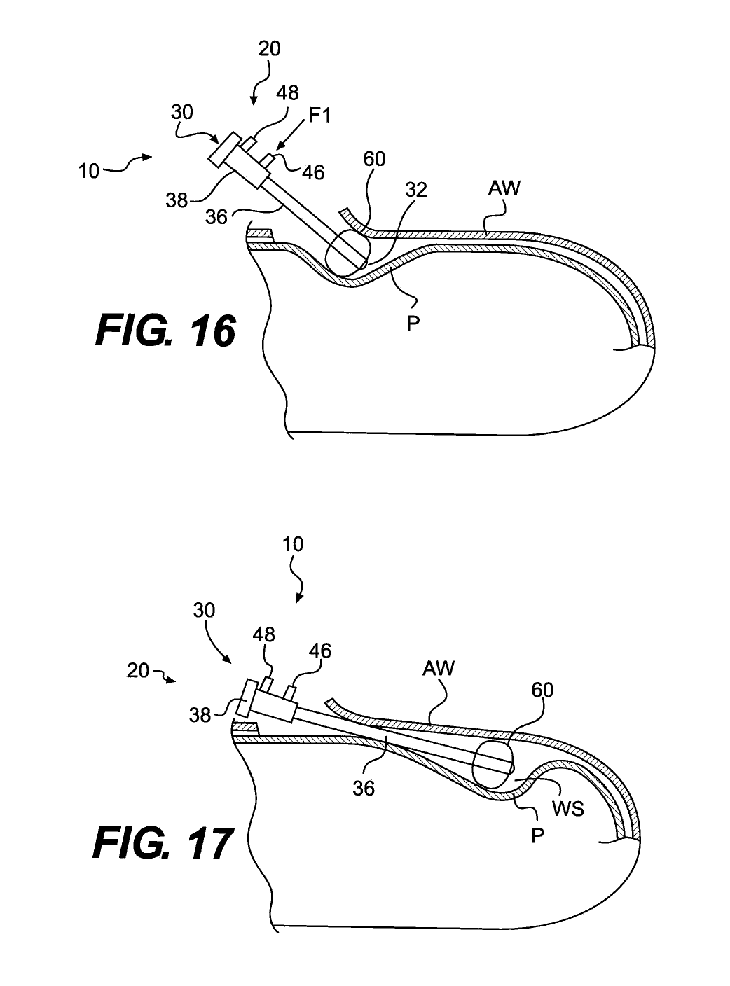

As shown in FIG. 16, inflation fluid may be directed to the first valve 46 (indicated by arrow F1), and the expandable member 60 may be at least partially expanded with the inflation fluid. The expandable member 60 expands between tissue layers, and progressively creates an increasing volume of retroperitoneal space between the peritoneum P and the adjacent tissues along the dissection tract.

The expandable member 60 may be at least partially expanded and at least partially collapsed a number of times to progressively separate the tissue layers. Also, the surgeon may use the optical device 94 described above to observe the dissection of the tissue layers and may decide whether and how much to expand and/or collapse the expandable member 60 based on the observations. The surgeon may also observe the dissection of the tissue to determine where to reposition the expandable member 60. The surgeon may also use tactile feedback from expanding, collapsing, and/or moving the expandable member 60 to determine whether and how much to expand and/or collapse the expandable member 60.

For example, after expanding the expandable member 60 the first time (thereby partially dissecting the tissue layers), the inflation fluid in the expandable member 60 may be vented and the expandable member 60 may return to an at least partially collapsed configuration. The portion of the peritoneum P that was separated by the expandable member 60 remains detached from the adjacent tissue layer. The dissection port 10, including the expandable member 60 in the collapsed configuration, may then be manipulated to advance the distal end 32 of the elongated member 30 to the limit of the created retroperitoneal space. The expandable member 60 may then be expanded again, thereby increasing the extent of the detached part of the peritoneum P. This “tunneling” process of collapsing the expandable member 60, advancing the distal end 32 of the elongate member 30 to the limit of the detached part of the peritoneum P, holding the distal end 32 of the elongate member 30 in position, and expanding the expandable member 60 again may be repeated until the created retroperitoneal space includes a desired work site. The expandable member 60 may also be at least partially expanded and collapsed to provide support to retract tissue away from a work site.

At any time during the steps shown in FIGS. 14-18 before or during the surgical procedure, the surgeon may insert and/or replace the insert component 80 inside the bore 40 in the port component 20, as shown in FIG. 18. The surgeon may also insert and/or replace working instruments 90 in the lumens 88 in the insert component 80 in the port component 20. The working instruments 90 may include end effectors 92, optical device 94, devices to assist in orienting or directing other working instruments 90, devices to dissect tissue, devices to assist in advancing or redirected the dissection port 10 or port component 20 (such as by pulling or retracting tissue, or initiating a new plane of dissection), or any of the other end effectors described above. The new plane of dissection may be located between tissue layers that are different from the tissue layers previously separated by the expansion of the expandable member 60. For example, the new plane of dissection may be initiated by at least partially collapsing the expandable member 60, pulling or pushing the port component 20 in a new direction, and expanding the expandable member 60. Alternatively, the new plane of dissection may be initiated by using a deflectable portion of a working instrument 90, e.g., with at least one of a blunt tip, an end effector 92 such as a grasper, or an expandable member on the working instrument 90. The insert component 80 and the working instruments 90 to be inserted may be selected based on the intended use for the working instruments 90 and the tasks to be completed.

As shown in FIG. 17, when the expandable member 60 is inflated, the expandable member 60 forms a seal to limit the escape of fluids, such as insufflation fluid and body fluids, from the working space WS within the patient by providing a substantially fluid-tight seal of the tunnel T with the tissue contacting an outer circumference of the expandable member 60. The seal may be used to block flow of any type of fluid, such as the insufflation fluid, water, saline, body fluids (e.g., gastric fluids, colonic fluids, blood, etc.), etc.

As shown in FIG. 18, the working space WS at the desired work site is then insufflated if necessary, by directing insufflation fluid (indicated by arrow F2) through the second valve 48 into the working space WS. As described above, the insufflation fluid may be directed from the second valve 48 either through the bore 40 in the elongate member 30 of the port component 20 or through the lumen 88 in the insert component 80 in the bore 40.

According to another embodiment, the dissection port 10 may be used for a transvaginal single-port sacrocolpopexy procedure. In such a procedure, the incision may be made in the vaginal wall using the dissection port 10, as in FIG. 14. Then, the expandable member 60 may be used to dissect tissue to create a tunnel outside and parallel to the vagina using the dissection port 10 with or without any appropriate insert component 80 and/or working instruments 90, as in FIGS. 15-18. After the retroperitoneal cavity is fully developed to allow access to the sacral promontory, a mesh or tissue graft may be delivered using one of the working instruments 90 inserted into a lumen 88 in the insert component 80 or through the bore 40 in the port component 20 (with or without an insert component 80 in the bore 40). One or more other working instruments 90 may be inserted through the lumen 88, another lumen 88, or through the bore 40 to attach the mesh to tissue in the body over the sacrum, pelvis, or vagina, and/or to observe the procedure.

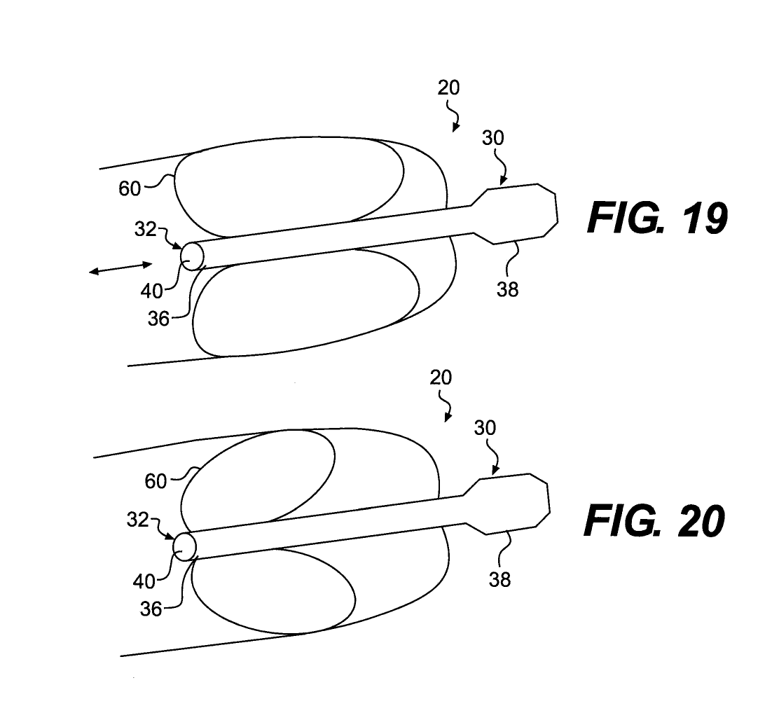

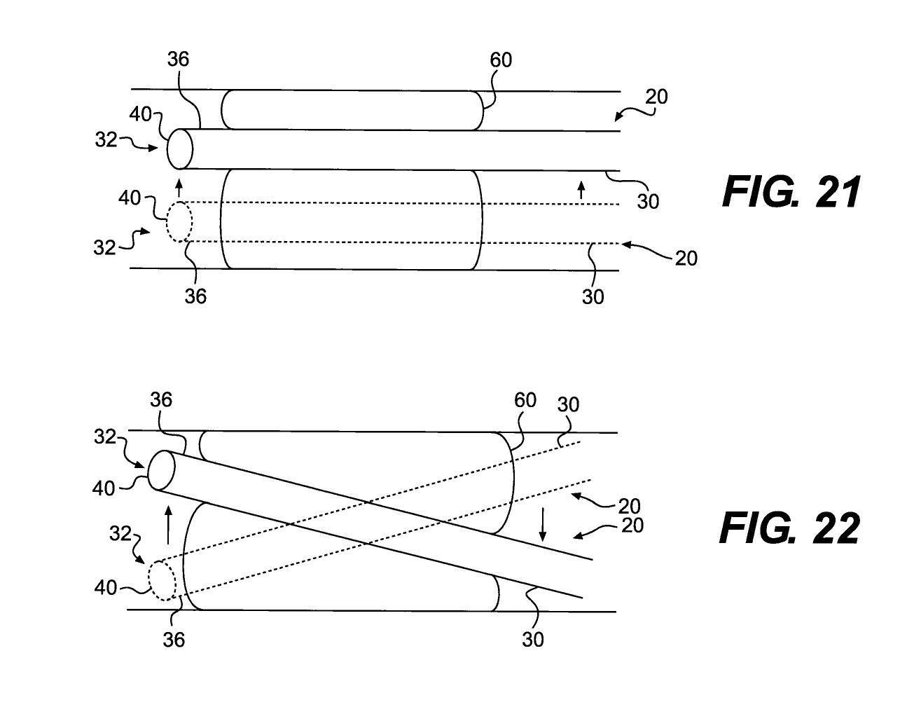

FIGS. 19-22 depict possible movement of the port component 20 and the expandable member 60 while the expandable member 60 maintains a seal inside the patient, as described above and shown in FIGS. 17 and 18. As shown in FIG. 19, when the expandable member 60 forms a seal inside the patient, the port component 20 may be moved longitudinally (as indicated by the arrows). FIG. 20 shows the expandable member 60 maintaining the seal inside the patient and the shape of the expandable member 60 when the port component 20 is moved longitudinally. FIG. 21 shows the expandable member 60 maintaining the seal inside the patient and the shape of the expandable member 60 when the port component 20 is moved laterally (as indicated by the arrows, with the elongate member 30 in a first position shown in dashed lines and in a second position shown in solid lines). FIG. 22 shows the expandable member 60 maintaining the seal inside the patient and the shape of the expandable member 60 when the port component 20 is moved angularly (as indicated by the arrows, with the elongate member 30 in a first position shown in dashed lines and in a second position shown in solid lines). Accordingly, the expandable member 60 may serve as a seal while allowing the port component 20 to move longitudinally, laterally, angularly, and/or rotationally.

According to an embodiment, the port component 20 may include more than one expandable member 60, e.g., of different shapes or sizes. For example, another expandable member 60 may be positioned on the cannula 36 proximal to the expandable member 60 shown in FIGS. 1-3 and 9-13. The expandable members 60 may be connected to the same inflation lumen 44 or to separate inflation lumens 44. If individually controlled, the expandable members 60 may be expanded and collapsed by the surgeon, e.g., sequentially, alternately, simultaneously, etc., to progressively dissect the tissue layers in the patient and/or to provide support for retracting tissue.

The dissection port 10 may be configured to provide multiple expandable members, such as the expandable member(s) 60 on the port component 20 and any expandable members that may be provided as end effectors 92 of the working instruments 90 inserted through the bore 40 and/or through the insert component 80. Each of these expandable members may dissect tissue, serve as a moveable seal, provide support to retract tissue near the opening OP (e.g., an incision through a tissue wall into a space, such as the retroperitoneal or peritoneal space), and/or provide a force (such as a radial force) against a body organ, a wall of a body organ, a body lumen, or any other type of body tissue (e.g., to cause the body lumen or body cavity to expand or otherwise move). For example, the expandable member 60 on the port component 20 may be used to dissect tissue and form the seal between the expandable member 60 and the tissue of the patient (as shown in FIGS. 17-22), and a second expandable member on a working instrument 90 inserted through the lumen 88 in the insert component 80 may be advanced distal to the expandable member 60 on the port component 20. The second expandable member may dissect tissue layers inside the patient, serve as an anchor, retract tissue, and/or form a second seal at another location inside the patient (in a similar manner as shown in FIGS. 17-22). Insufflation fluid may be provided, as described above and shown in FIG. 18, and the two expandable members providing seals may be used to contain the insufflation fluid in the working space WS.

The dissection port 10 is capable of performing multiple tasks without having to remove the port component 20 from inside the patient. For example, the expandable member 60 of the dissection port 10 may dissect tissue (as shown in FIGS. 15 and 16) and provide a seal between the expandable member 60 and the tissue of the patient (as shown in FIGS. 17-22) without having to remove the cannula 36 or the port component 20 from inside the patient and without having to use any additional expandable members. Furthermore, while maintaining the seal between the expandable member 60 and the tissue of the patient and without having to remove the port component 20 from the patient, the insert component 80 may be replaced with a different insert component 80, e.g., to provide a different configuration of lumens 88 based on the intended tasks to be performed and the working instruments 90 to be used, and/or the working instruments 90 may be replaced with different working instruments 90. Moreover, while maintaining the seal between the expandable member 60 and the tissue of the patient and without having to remove the port component 20 from the patient, the expandable member 60, insert component 80, and/or working instrument 90 may be used to retract, move, or push/pull against tissue or body organs inside the patient with or without insufflating the working space WS. The expandable member 60 in its expanded configuration, when positioned between two dissected tissue layers, may not only anchor the cannula 36 and the port component 20 to the patient but may also retract the dissected tissue layers by a desired amount.

Although multiple expandable members may be provided, the dissection port 10 may include the single expandable member 60 for dissecting tissue, providing the seal, and/or anchoring the port component 20. Multiple expandable members may be provided, but are not required, for these functions. Furthermore, regardless of the number of expandable members provided, the port component 20 does not have to be removed from the body and/or replaced in order to perform all of these functions (e.g., dissection, sealing, and/or anchoring). Instead of having to replace the port component 20 or the dissection port 10 when a different configuration of lumens 88 is preferred for a particular task, the insert component 80 may be removed and replaced. By simply replacing the insert component 80, the position of the port component 20 inside the patient may be maintained and an insufflation seal may be maintained by the expandable member 60. As a result, there may be less trauma to the patient and fewer complications.

Since the insert component 80 includes multiple lumens 88, the dissection port 10 may be a single port multi-channel device. The dissection port 10 is a “single port” device since it may be inserted into a single opening OP (e.g., through a natural orifice or a single incision) in the body to operate inside the body. With only a single entry point, there may be reduced morbidity, increased cosmesis, less post-operative pain, less blood loss, faster recovery, fewer complications, etc. The dissection port 10 may also be a “multi-channel” device since one of the interchangeable insert components 80 may include multiple lumens 88 or channels through which the working instruments 90 may be inserted. With multiple channels, the surgeon may be able to accomplish a wide variety of tasks using multiple working instruments 90 at one time. Thus, the dissection port 10 may provide the advantages of a single port device, a device with multiple channels, a tissue plane dissection device, and a device for creating an insufflation seal.

The dissection port 10 set forth herein may be made of a suitable biocompatible material. Any aspect set forth in any embodiment may be used with any other embodiment set forth herein. Every device and apparatus set forth herein may be used in any suitable medical procedure, may be advanced through any suitable body lumen and body cavity, and may be used to access tissue from any suitable body portion. For example, the apparatuses and methods described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only. The following disclosure identifies some other exemplary embodiments.

In some embodiments, a device to provide working access to a surgical site in a patient may include a port component including an elongate member configured to pass through an opening in the patient. The elongate member may include a bore extending between a distal end and a proximal end of the elongate member. The port component may also include an expandable member disposed on the elongate member. The device may also include an insert component configured to be removably received inside the bore in the elongate member of the port component. The insert component may include at least one lumen.

In some embodiments, the opening in the patient may be through a natural orifice.

In some embodiments, the opening in the patient may be one of a mouth, anus, nose, urethra, umbilicus, and vagina of the patient.

In some embodiments, the opening in the patient may be an incision in epithelium of the patient.

In some embodiments, the insert component may be configured to extend through the expandable member.

In some embodiments, the expandable member may be configured to expand in at least one of a radial, lateral, or longitudinal direction with respect to a longitudinal axis of the elongate member and a longitudinal axis of the insert component.

In some embodiments, the expandable member may be configured to be inserted into the patient so that the expandable member is capable of separating tissue layers in the patient when expanded.

In some embodiments, the elongate member may further include a first valve disposed proximal to the expandable member and an inflation lumen fluidly connected to the expandable member and the first valve.

In some embodiments, the elongate member may further include a second valve disposed proximal to the expandable member, and the second valve may be configured to fluidly connect to the bore or the lumen in the insert component that extends to a distal end of the insert component.

In some embodiments, the second valve and at least one of the bore and the lumen in the insert component may be configured to receive an insufflation fluid and direct the insufflation fluid into a working space between tissue layers in the patient.

In some embodiments, the elongate member may further include a second valve disposed proximal to the expandable member, and the second valve may be configured to fluidly connect to a groove on an outer surface of the insert component. The groove may extend to a distal end of the insert component.

In some embodiments, at least one of the bore in the elongate member and the lumen in the insert component may be configured to receive an insufflation fluid and direct the insufflation fluid toward a working space between tissue layers in the patient.

In some embodiments, the expandable member may be configured to form a seal between the expandable member and tissue of the patient while the insufflation fluid is directed toward the working space.

In some embodiments, the elongate member may be capable of being repositioned in at least one of a rotational, longitudinal, angular, or lateral direction while maintaining the seal between the expandable member and the tissue of the patient.

In some embodiments, the lumen in the insert component may be configured to removably receive a working instrument configured to extend through the respective lumen and exit through a distal end of the lumen.

In some embodiments, the working instrument may include an end effector configured to pass through the respective lumen and exit through the distal end of the respective lumen.

In some embodiments, the expandable member on the elongate member may be a first expandable member, and the working instrument may include a second expandable member disposed on the working instrument.

In some embodiments, the second expandable member may be moveable inside the patient when the first expandable member abuts tissue of the patient to form a seal.

In some embodiments, the working instrument may be a first working instrument, and the first working instrument may include a lumen configured to removably receive a second working instrument.

In some embodiments, the lumen in the insert component may be configured to receive a plurality of working instruments.

In some embodiments, the insert component may be a first insert component, and the device may further include a second insert component removably received inside the lumen in the first insert component.

In some embodiments, the insert component may include a flange disposed on a proximal end of the insert component, and the flange may be configured to abut the proximal end of the elongate member.

In some embodiments, the insert component may include at least one seal on a proximal end of the insert component, and the seal may be capable of restricting a flow of fluid out of the lumen of the insert component.

In some embodiments, only one expandable member is disposed on the elongate member.

In some embodiments, the device may further include an optical device disposed on, in, or through the insert component near a distal end of the insert component.

In some embodiments, the insert component may be one of a plurality of interchangeable insert components, and each insert component may have a different configuration of zero, one, or more than one lumen.

In some embodiments, the insert component may be removable from the elongate member when the expandable member is in the expanded configuration.

In some embodiments, the expandable member may be toroidal when expanded and may have an outer profile that is circular, oval, elliptical, teardrop-shaped, triangular, or square.

In some embodiments, the expandable member may have an axis that is parallel to a longitudinal axis of the elongate member and a longitudinal axis of the insert component.

In some embodiments, the elongate member may include only one bore that extends through the distal end of the elongate member.

In some embodiments, the insert component may include a plurality of lumens, and each lumen may be configured to removably receive at least one working instrument configured to extend through the respective lumen and exit through a distal end of the lumen.

In some embodiments, at least one of the elongate member or the insert component may be flexible or malleable.

In some embodiments, a method to provide working port access to a surgical site in a patient may include advancing an expandable member in a collapsed configuration and an elongate member through an opening in the patient. The expandable member may be disposed on the elongate member. The method may also include expanding the expandable member to an expanded configuration to separate tissue layers in the patient and inserting a removable insert component including at least one lumen into a bore in the elongate member.

In some embodiments, the method may further include positioning the expandable member in the expanded configuration to form a seal between the expandable member and tissue of the patient.

In some embodiments, the method may further include directing insufflation fluid through at least one of lumen in the insert component and the bore in the elongate member so that the insufflation fluid forms a working space in the patient when the expandable member forms the seal between the expandable member and the tissue of the patient.

In some embodiments, the expandable member in the expanded configuration may be positioned to form the seal after expanding the expandable member and without removing the expandable member from inside the patient.

In some embodiments, the seal may be formed between a circumference of the expandable member and the tissue of the patient.

In some embodiments, the method may further include repositioning the elongate member while maintaining the seal between the expandable member and tissue of the patient.

In some embodiments, the opening in the patient may be through a natural orifice.

In some embodiments, the opening in the patient may be one of a mouth, anus, nose, urethra, umbilicus, and vagina of the patient.

In some embodiments, the method may further include making an incision in epithelium of the patient, and the incision may be the opening in the patient through which the elongate member is advanced.

In some embodiments, the elongate member may include only one bore extending through a distal end of the elongate member.

In some embodiments, the method may further include inserting an obturator into the elongate member, and using the obturator and the expandable member in the expanded configuration to separate the tissue layers in the patient.

In some embodiments, the obturator may include a rounded tip on a distal end of the obturator positioned distal to the expandable member, and the rounded tip of the obturator may assist in separating the tissues layers in the patient.

In some embodiments, the method may further include removing the obturator from the elongate member before inserting the insert component into the bore in the elongate member.

In some embodiments, the insert component may include a plurality of lumens extending between a distal end of the elongate member and a proximal end of the elongate member.

In some embodiments, the method may further include inserting a working instrument into the lumen in the insert component, and the working instrument may include an end effector at a distal end of the working instrument.

In some embodiments, the working instrument may be a first working instrument, and the method may further include inserting a second working instrument or supplying a fluid through a lumen in the first working instrument.

In some embodiments, the expandable member on the elongate member may be a first expandable member, and the working instrument may include a second expandable member disposed on the working instrument. The method described above may further include positioning the first expandable member in the expanded configuration to form a seal between the first expandable member and tissue of the patient, and advancing the second expandable member distal to the first expandable member and expanding the second expandable member while the first expandable member forms the seal.

In some embodiments, the second expandable member may form a seal with tissue contacting the second expandable member.

In some embodiments, the second expandable member may be expanded to separate tissue layers in the patient.

In some embodiments, the insert component may slide into the bore in the elongate member until a flange on a proximal end of the insert component abuts a proximal end of the elongate member.

In some embodiments, the method may further include selecting the insert component to place into the bore of the elongate member based on the configuration of at least one lumen in the insert component.

In some embodiments, the insert component may be selected based on an intended use for at least one lumen in the insert component.

In some embodiments, the insert component may be selected from a plurality of interchangeable insert components, and each interchangeable insert component may have a different configuration of zero, one, or more than one lumen.

In some embodiments, the method may further include at least partially collapsing the expandable member to an at least partially collapsed configuration after expanding the expandable member, moving the expandable member after at least partially collapsing the expandable member, and expanding the expandable member to further separate tissue layers in the patient.

In some embodiments, the method may further include observing the separating of the tissue layers.

In some embodiments, the method may further include inserting a working instrument including an optical device for observing the separating of the tissue layers into at least one lumen in the insert component.

In some embodiments, the method may further include transmitting an image signal from an optical device located near a distal end of the insert component to observe the separating of the tissue layers.

In some embodiments, the image signal may be transmitted to a user wirelessly or with a wire embedded into the insert component.

In some embodiments, the expandable member may be repositioned in at least one of a rotational, longitudinal, lateral, or angular direction.

In some embodiments, the method may further include using a seal to restrict a flow of fluid out of at least one lumen of the insert component.

In some embodiments, the method may further include using the seal to restrict the flow of fluid out of at least one lumen of the insert component independent of whether a working instrument is disposed in at least one lumen.

In some embodiments, expanding the expandable member to the expanded configuration may create a first plane of dissection, and the method described above may further include inserting an insert component and/or a working instrument into the lumen in the insert component and positioning the working instrument to initiate a second plane of dissection.

In some embodiments, expanding the expandable member to the expanded configuration may create a first plane of dissection, and the method described above may further include at least partially collapsing the expandable member to an at least partially collapsed configuration after expanding the expandable member, repositioning the expandable member, and expanding the expandable member to initiate a second plane of dissection.

In some embodiments, a method to provide working port access to a surgical site in a patient may include advancing an expandable member in a collapsed configuration through an opening in the patient. The expandable member may be disposed on an elongate member. The method may also include expanding the expandable member to an expanded configuration, positioning the expandable member in the expanded configuration to abut tissue of the patient to form a seal between the expandable member and the tissue of the patient, and directing insufflation fluid through at least one of the elongate member and an insert component that is removably received in the elongate member so that the insufflation fluid forms and/or maintains a working space in the patient.

In some embodiments, the method may further include removing the insert component from the elongate member after forming the working space in the patient, and replacing the insert component with a different insert component.

In some embodiments, the insert component may be removed without removing the expandable member from inside the patient.

In some embodiments, the method may further include inserting a working instrument into a lumen in the insert component.

In some embodiments, the working instrument may be positioned in the lumen while insufflation fluid is supplied.

In some embodiments, the method may further include making an incision in epithelium of the patient, and the incision may be the opening in the patient through which the expandable member is advanced.

In some embodiments, the method may further include removing the insert component and placing another insert component having a different configuration of one or more lumens into the elongate member.

In some embodiments, the method may further include observing the working space in the patient.

In some embodiments, the method may further include inserting a working instrument including an optical device for observing the working space into a lumen in the insert component.

In some embodiments, the method may further include transmitting an image signal from an optical device located near a distal end of the insert component to observe the working space.

In some embodiments, a method to provide working port access to a surgical site in a patient may include advancing an expandable member in a collapsed configuration and an elongate member through an opening in the patient. The expandable member may be disposed on the elongate member. The method may also include expanding the expandable member to an expanded configuration to separate tissue layers in the patient, removing a first insert component from inside of the elongate member without removing the expandable member from inside the patient, and inserting a second insert component into the elongate member when the expandable member is inside the patient. The first insert component may have a different structure than the second insert component.

In some embodiments, the first and second insert components may have a different configuration of zero, one, or more than one lumen.

In some embodiments, at least one of the first and second insert components may include a blunt distal tip.

In some embodiments, the method may further include inserting at least one working instrument into at least one lumen in at least one of the first and second insert components.

In some embodiments, the method may further include transmitting an image signal from an optical device located near a distal end of at least one of the first and second insert components.

What is claimed is:

1. A device for providing working access to a surgical site in a patient, the device comprising:
a port component including:
an elongate member configured to pass through an opening in the patient, the elongate member including a bore extending between a distal end and a proximal end of the elongate member, and
an expandable member disposed on the elongate member; and
an insert component configured to be removably received inside the bore in the elongate member of the port component, the insert component including at least one lumen.

2. The device of claim 1, wherein the opening in the patient is through a natural orifice or an incision in the patient.

3. The device of claim 1, wherein the insert component is configured to extend through the expandable member.

4. The device of claim 1, wherein the expandable member is configured to expand in at least one of a radial, lateral, or longitudinal direction with respect to a longitudinal axis of the elongate member and a longitudinal axis of the insert component.

5. The device of claim 1, wherein the expandable member is configured to be inserted into the patient so that the expandable member is capable of separating tissue layers in the patient when expanded.

6. The device of claim 1, wherein the elongate member further includes a first valve disposed proximal to the expandable member and an inflation lumen fluidly connected to the expandable member and the first valve.

7. The device of claim 6, wherein the elongate member further includes a second valve disposed proximal to the expandable member, the second valve being configured to fluidly connect to the bore, the lumen in the insert component that extends to a distal end of the insert component, or a groove extending to a distal end of the insert component on an outer surface of the insert component.

8. The device of claim 1, wherein at least one of the bore in the elongate member and the lumen in the insert component are configured to receive an insufflation fluid and direct the insufflation fluid toward a working space between tissue layers in the patient.

9. The device of claim 8, wherein:
the expandable member is configured to form a seal between the expandable member and tissue of the patient while the insufflation fluid is directed toward the working space; and
the elongate member is capable of being repositioned in at least one of a rotational, longitudinal, angular, or lateral direction while maintaining the seal between the expandable member and the tissue of the patient.

10. The device of claim 1, wherein the lumen in the insert component is configured to removably receive at least one working instrument configured to extend through the respective lumen and exit through a distal end of the lumen.

11. The device of claim 10, wherein:
the expandable member on the elongate member is a first expandable member, and the at least one working instrument includes a second expandable member disposed on the at least one working instrument; and
the second expandable member is moveable inside the patient when the first expandable member abuts tissue of the patient to form a seal.

12. The device of claim 1, wherein:
the insert component includes a flange disposed on a proximal end of the insert component, the flange being configured to abut the proximal end of the elongate member; and
the insert component includes at least one seal on a proximal end of the insert component, the seal being capable of restricting a flow of fluid out of the lumen of the insert component.

13. The device of claim 1, further including an optical device disposed on, in, or through the insert component near a distal end of the insert component.

14. The device of claim 1, wherein the insert component is one of a plurality of interchangeable insert components, and each insert component has a different configuration of zero, one, or more than one lumen.

15. The device of claim 1, wherein the expandable member is toroidal when expanded and has an outer profile that is circular, oval, elliptical, teardrop-shaped, triangular, or square.

16. The device of claim 1, wherein at least one of the elongate member or the insert component is flexible or malleable.

17. A method for providing working port access to a surgical site in a patient, the method comprising:
advancing an expandable member in a collapsed configuration and an elongate member through an opening in the patient, the expandable member being disposed on the elongate member;
expanding the expandable member to an expanded configuration to separate tissue layers in the patent; and
inserting a removable insert component including at least one lumen into a bore in the elongate member.

18. The method of claim 17, wherein the removable insert component is a first insert component, and the method further includes:
removing the first insert component from inside the bore in the elongate member without removing the expandable member from inside the patient; and
inserting a second insert component into the bore in the elongate member when the expandable member is inside the patient, the first insert component having a different structure than the second insert component.

19. The method of claim 17, further including inserting a working instrument into the lumen in the insert component, the working instrument including an end effector at a distal end of the working instrument.

20. A method of for providing working access to a surgical site in a patient, the method comprising:
advancing an expandable member in a collapsed configuration through an opening in the patient, the expandable member being disposed on an elongate member;
expanding the expandable member to an expanded configuration;
positioning the expandable member in the expanded configuration to abut tissue of the patient to form a seal between the expandable member and the tissue of the patient; and
directing insufflation fluid through at least one of the elongate member and an insert component that is removably received in the elongate member so that the insufflation fluid forms a working space in the patient.
